(19) United States
(12) Patent Application Publication
(10) Pub. No.: US 2008/0199409 A1
(43) Pub. Date: Aug. 21, 2008

(54) Effervescent Acidified Rehydration Preparations

(75) Inventors: Michael L. Nelson, Waconia, MN (US); Martin J. Nelson, Scottsdale, AZ (US)

Correspondence Address: NIKOLAI & MERSEREAU, P.A., 900 SECOND AVENUE SOUTH, SUITE 820, MINNEAPOLIS, MN, 55402, US

(73) Assignee: Tech Mix Inc., Stewart, MN (US)

(21) Appl. No.: 11/675,253

(22) Filed: Feb. 15, 2007

(51) Int. Cl. A61K 9/10

(52) U.S. Cl. 424/44; 424/442

(57) ABSTRACT

An effervescent acidified rehydration preparation for administration in the drinking water of mammals and poultry includes an amount of a dry generally water soluble acidified electrolyte composition for treatment of dehydration in animals including mammals and poultry by oral administration in the drinking water of animals of interest and an amount of a pH balanced effervescent composition including an amount of one or more compatible water soluble organic acid species and an amount of one or more water soluble effervescent agents wherein effervescence occurs immediately upon the rehydration preparation being added to water.

### BACKGROUND OF THE INVENTION

I. Field of the Invention

The present invention relates generally to orally administered preparations for treating dehydration in animals including livestock and poultry and, more specifically, to dry effervescent, acidified rehydration preparations having enhanced suspension properties when added to drinking water of selected animals.

II. Related Art

It is known that water represents over 50% of the body weight of mature animals such as porcine, bovine, ovine, equine and other species of livestock, as well as avian species including poultry. In addition, young animals have a much higher percentage of water in their bodies than mature animals. For example, the body weight of young piglets is almost 75% water. Fluids are balanced in the body in what may be described as three basic compartments and electrolytes in the fluids and their corresponding osmotic pressure relations operate to maintain body fluids at proper levels. This involves a balance among the three fluid compartments, namely, intracellular fluid in the cells, interstitial fluid in tissue outside the cells and blood or vascular fluids. Of course, without adequate water and electrolytes, a body cannot maintain proper fluid balance between the three compartments which is necessary to maintain proper health.

Dehydration may severely affect fluid/electrolyte balances and has become a major significant stress factor that impacts the profitability and efficiency of livestock and poultry production. For example, animals that dehydrate or shrink over 10% of their total body weight have a much higher mortality rate than animals that do not shrink or dehydrate. Researchers and veterinarians who encounter shrink symptoms exceeding 10% of body weight recommend intravenous therapy including fluids, electrolytes and energy boosters until normal body weights are again obtained. Animals suffering with this degree of dehydration seldom consume feed or dry matter which also contributes to significantly higher mortality rates. Animals that do not recover quickly from shrink or dehydration symptoms amounting to over 10% of body weight not only have higher mortality rates, but also have significantly reduced ability to return to normal levels of production and performance.

Animals may suffer some degree of dehydration or shrink as a result of disease or environmental stresses such as severe weather changes or changes of environment, shipping, moving or changes of management in feeding related to weaning, housing and handling. In this regard, it has been found that animals shipped and restricted from feed and water during shipment for 12-36 hours will have dehydration or shrink rates ranging from 3-7.5%. If these animals are given the appropriate oral rehydration product in their drinking water, they will continue to maintain some degree of normal feed and water intake thereby reducing the chances of severe dehydration, i.e., over 10% or more. In addition, when these animals encounter conditions or factors contributing to shrink and dehydration, but are given appropriate rehydration products immediately or as soon as possible upon being exposed to dehydrating factors, they return to normal production rates more quickly and do not show signs of extended shrink or impaired production.

It is known to provide oral rehydration products to prevent and reverse the effects of dehydration and shrinkage in livestock, poultry and even pets. Compositions for treating dehydration in livestock mammals, for example, are found in U.S. Pat. No. 4,839,347, which is assigned to the same assignee as the present invention. The entirety of that document is deemed incorporated herein by reference for any purpose.

It has also been found that animal oral rehydration products are far more effective if the ingested pH of the material is close to the pH of the digestive tract of the animal involved. Most stomach and gastric enzymes have little ability to digest food until the stomach acids have acidified the ingested food or feed from the original pH of 7.0 or more to a pH of 4.0-5.0. This acidifying action is found to be a normal digestive process in humans and animals of similar digestive tracts such as dogs, pigs, horses, poultry and cats. Calves prior to weaning have a similar digestive process in their stomach to digest milk. Calves after weaning develop a rumen to break down grains and roughages in their large or fore stomach compartment. Adult cattle have a similar acidifying digestive action in their fourth stomach compartment or abomasums as do simple stomach animals such as pigs, horses and dogs.

It has further been found that pH readings of stomach linings of such animals range from about 3.0-about 4.0 and, accordingly, it has been found that an oral rehydration product, when added to regular drinking water with pH ranges of 6.0-8.0 that reduces or changes the pH of the drinking water to a range generally from about 3.0-about 4.0, is most effective. At the proper pH, the acidifying action promotes quick utilization of the oral rehydration benefits of the electrolytes and energy sources. Further benefits of acidifying oral rehydration products include their ability to reduce the growth of pathogens such as E. Coli and Salmonella. These bacterial pathogens have virtually no ability to grow and flourish in pH ranges from 3 to 5.

In this regard, oral rehydration products which mimic the pH of digestive tracts of the animals sought to be treated are more effective. Such products have also been made available in powder or tablet forms designed to be added to the drinking water of the animals.

While success has been achieved utilizing prior products, under field and pen conditions many water sources or containers commonly used for water consumption by livestock and poultry consist of large stock tanks, pails or containers that hold drinking water in volumes ranging from 5 to several hundred gallons of water. When rehydration products are added to these containers, the products must be stirred and mixed vigorously by hand or using mixing tools such as paddles for extensive periods of time to assure that the materials that dissolve are completely dissolved and that a uniform suspension of active agents which do not completely dissolve in the drinking water is provided. Even the most soluble of these products have ingredients that will tend to migrate or settle at the bottom of the container, thereby prohibiting the complete administration of the formulation in a uniform manner to an animal drinking from the container.

Thus, while much progress has been made, there remains a definite need to provide such a rehydration product which is more readily dispersed in and thoroughly mixed with the water to provide a more uniformly suspended re-hydrating formula than was formerly available.

### SUMMARY OF THE INVENTION

By means of the present invention, there is provided an effervescent acid rehydration product which is much easier to uniformly administer in drinking water under field conditions to provide maximum rehydration benefits to animals and poultry. The invention makes it easier to provide a thoroughly mixed (uniformly suspended) formula and facilitates a better overall result with both mammals and poultry. The effervescent oral rehydration formula of the present invention maintains a uniform condition in drinking water for a far longer period than water of prior rehydration formulas.

In accordance with embodiments of the invention, significant effervescence has been added to animal rehydration powders and tablets. The effervescence has been found to impart natural mixing and suspension enhancing qualities to the material when the powder or tablets are added to drinking water. This eliminates the need for mixing equipment in the field or other inconvenient places.

Embodiments of the effervescent formulas of the present invention may be in a form that combines known acidifying oral rehydration products, or newly formulated products, with pH balanced or buffered effervescent material combinations to produce dissolvable oral rehydration products in powder and tablet form which dissolve rapidly in drinking water and form a more uniform and long-lasting suspension of active agents in the drinking water. The pH balance is designed to minimize the effect of the effervescent materials on the pH of the drinking water established by the oral rehydration product to which effervescence is added.

Effervescent compositions or systems include one or more organic acids which are compatible both with the ingredient oral rehydration formula and are readily ingested by the animal species of interest combined with one or more effervescent agents. The systems include an amount of one or more acids selected from compatible organic acids including citric acid, tartaric acid, malic acid, fumaric acid, and the like, which are generally solid or crystalline at room temperature and which are generally freely soluble in water. The one or more acids are combined with one or more soluble effervescent agent species including carbonates and bicarbonates such as sodium bicarbonate, sodium carbonate, potassium carbonate, magnesium carbonate, calcium bicarbonate, potassium bicarbonate, or the like.

To maintain a drinking water pH generally in the 3.0 to 4.0 range, it has been found that generally an effervescent agent to acid ratio in the effervescent additive composition of about 2:1 (weight) works well.

### BRIEF DESCRIPTION OF THE DRAWINGS

In the drawings wherein like numerals designate like parts throughout the same:

FIGS. 1a-1n picture a comparison of progressive incorporation of an acidified electrolyte rehydration preparation in water without externally applied stirring comparing the preparation with and without the addition of an effervescent composition in accordance with the invention.

### DETAILED DESCRIPTION

The following detailed description presents certain embodiments which illustrate the concept of the present invention. These embodiments are presented as examples and are not intended to limit the scope of the invention in any manner as variation may occur to those skilled in the art which are within the scope of the inventive concepts.

An important aspect of the present invention is that it greatly facilitates proper administration of rehydration preparations to all types of mammals, including livestock and poultry. It is especially beneficial in situations where amounts of solid rehydration preparations are added to drinking water at locations where additional stirring is difficult or unavailable as is often the case where such animals are under stress and likely to dehydrate. Such beneficial oral rehydration products are generally marketed and available in powder and tablet forms to be later dissolved in the drinking water of the animals or poultry of interest.

While these products are generally highly soluble, certain of the ingredients of the composition remain in suspension rather than completely dissolving. Portions of these products do tend to migrate or settle at the bottom of the drinking container without external stirring. In accordance with the present invention, however, it has been found that the addition of an effervescent composition not only speeds dissolution of the material in the intended drinking water, but it also quickly allows the formation of a more uniform suspension of active ingredients in the drinking water which continues for a sufficient time for the material to be ingested by the animals of interest.

The effervescent compositions of the present invention are designed to be incorporated in new or existing oral rehydration product powders and tablets. The effervescent compositions are preferably formulated so as not to affect the desired pH of the drinking water as determined by the rehydration preparation to which the effervescent composition has been added. Thus, the effervescent rehydration preparations are preferably acidified to a pH in accordance with the digestive tract of the species for which the particular formula is designed, which is generally from about 3.0-about 4.0. Thus, in accordance with the invention, an effervescent action is added to the rehydration products without a significant effect on the pH range of the drinking water. This is accomplished by balancing one or more effervescent agents with one or more compatible organic acids.

In this regard, it has generally been found that a combination of one part (weight) compatible soluble organic acid and two parts (weight) effervescent agent in the effervescent composition generally has little effect on the pH of drinking water when added to acidified rehydration preparations such as those available from TechMix, Inc. of Edina, Minn., the Assignee of the present application. These include a family of acidified supplement products sold as powders and tablets for addition to the drinking water of animals to prevent dehydration and restore energy following environment, nutritional or management challenges. These products are sold under the trademark BlueLite®.

Several effervescent formulas which have been tested with respect to effective solubilizing and suspension and acceptably low effect on drinking water pH are established by acidified rehydration products as follows:

| | | |
|---|---|---|
| 1 | Citric acid | 33% |
| | Sodium Bicarbonate | 67% |
| | | 100% |
| 2 | Citric acid | 23% |
| | Tartaric acid | 10% |
| | Sodium Bicarbonate | 50% |
| | Sodium Carbonate | 10% |
| | Potassium Carbonate | 7% |
| | | 100% |
| 3 | Citric acid | 18% |
| | Tartaric acid | 10% |
| | Malic acid | 5% |
| | Sodium Bicarbonate | 46% |
| | Sodium Carbonate | 10% |
| | Potassium Carbonate | 7% |
| | Magnesium Carbonate | 4% |
| | | 100% |
| 4 | Citric acid | 17% |
| | Tartaric acid | 8% |
| | Malic acid | 5% |
| | Sodium Bisulfate | 3% |
| | Sodium Bicarbonate | 41% |
| | Sodium Carbonate | 15% |
| | Potassium Carbonate | 7% |
| | Magnesium Carbonate | 4% |
| | | 100% |

Of course, other compatible acids such as fumaric acids and additional bases such as potassium bicarbonate and calcium bicarbonate could also be used. It has been found that the effervescent compositions can be added to the acidified rehydration preparations in varying amounts ranging from 1% up to about 30% depending on the application as long as the proper pH balance is maintained in the effervescent composition.

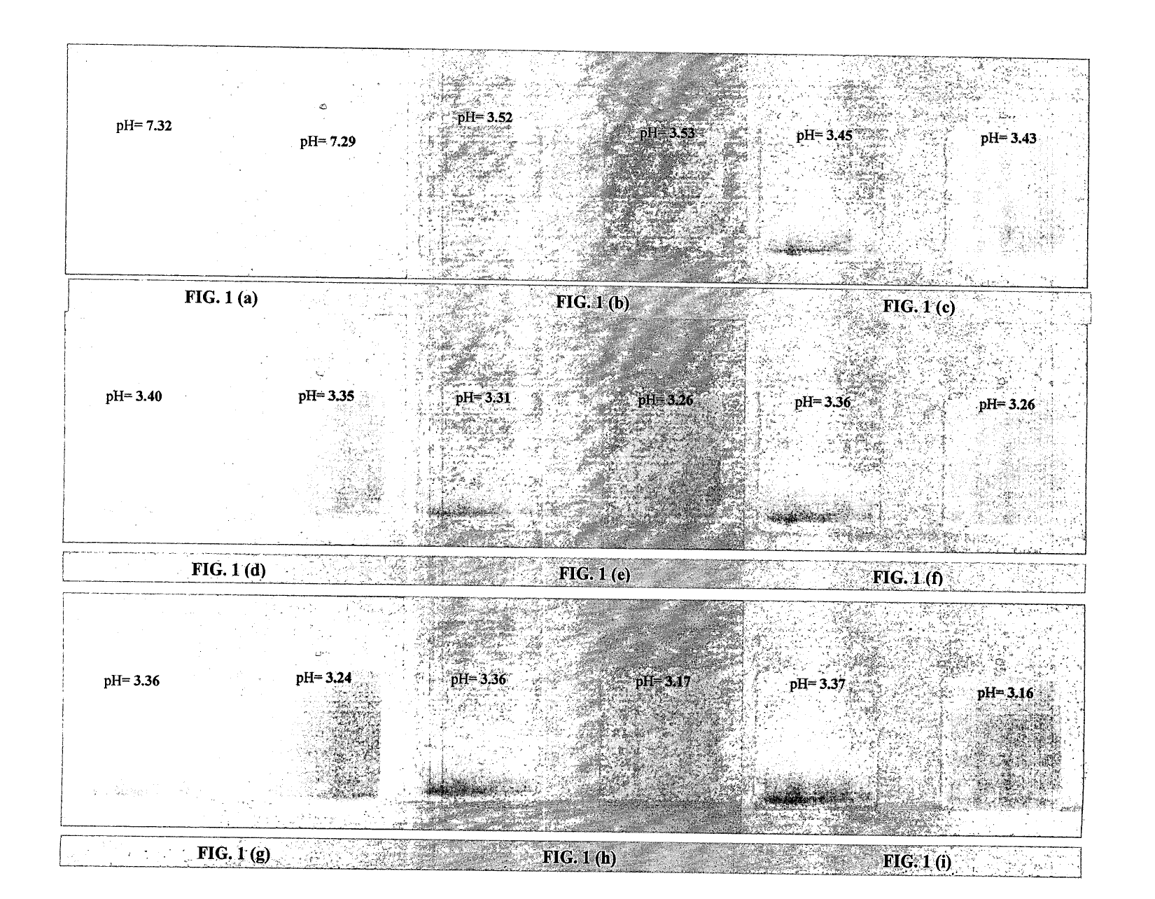

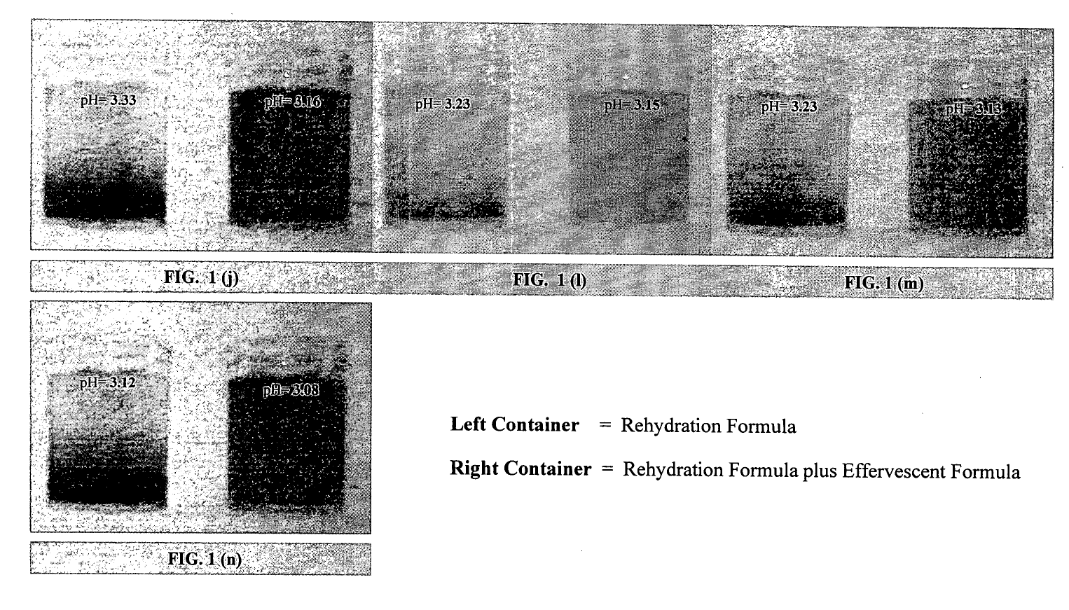

The improved mixing effect of the effervescent composition is illustrated in FIGS. 1a-1m and is presented in Table I which compares the addition of a Swine BlueLITE® rehydration preparation (present formula) with and without the addition of 1%-30% (weight) of a pH balanced effervescent composition as in No. 1 (above).

TABLE I

| | pH Measurements | Initial pH of water | |
|---|---|---|---|
| | Initial pH of | with no SWINE | |
| | water with no | BLUELITE ® and no | Drawing |
| | SWINE BLUELITE ® | EFFERVESCENT FORMULA | Reference |
| | 7.32 | 7.29 | |
| | | pH of water with | |
| | pH of water with | SWINE BLUELITE ® and | |
| | SWINE BLUELITE ® | EFFERVESCENT FORMULA | |
| pH after 10 seconds | 3.52 | 3.53 | FIG. 1(b) |
| with no stirring or | | | |
| mixing action | | | |
| pH after 20 seconds | 3.45 | 3.43 | FIG. 1(c) |
| with no stirring or | | | |
| mixing action | | | |
| pH after 30 seconds | 3.40 | 3.35 | FIG. 1(d) |
| with no stirring or | | | |
| mixing action | | | |
| pH after 40 seconds | 3.31 | 3.26 | FIG. 1(e) |
| with no stirring or | | | |
| mixing action | | | |
| pH after 50 seconds | 3.36 | 3.26 | FIG. 1(f) |
| with no stirring or | | | |
| mixing action | | | |
| pH after 1 minute with | 3.36 | 3.24 | FIG. 1(g) |
| no stirring or mixing | | | |
| action | | | |
| pH after 1½ minutes | 3.36 | 3.17 | FIG. 1(h) |
| with no stirring or | | | |
| mixing action | | | |
| pH after 2.0 minutes | 3.37 | 3.16 | FIG. 1(i) |
| with no stirring or | | | |
| mixing action | | | |
| pH after 3.0 minutes | 3.33 | 3.16 | FIG. 1(j) |
| with no stirring or | | | |
| mixing action | | | |
| pH after 4.0 minutes | 3.23 | 3.15 | FIG. 1(l) |
| with no stirring or | | | |
| mixing action | | | |
| pH after 5.0 minutes | 3.23 | 3.13 | FIG. 1(m) |
| with no stirring or | | | |
| mixing action | | | |
| pH after 10.0 minutes | 3.12 | 3.08 | FIG. 1(n) |
| with no stirring or | | | |
| mixing action | | | |

In this regard, note that, even after 10 seconds (FIG. 1b) the preparation containing the effervescent composition already exhibits uniform distribution in the drinking water container whereas the unstirred swine formula without the effervescent composition retains a somewhat settled or layered structure throughout. It should be noted further that the pH of the preparation, including the effervescent composition, very closely parallels that of the drinking water including the rehydration preparation, but without the effervescent composition.

The above table includes a swine formula known as Swine BlueLITE® available as of the date of this application.

In the laboratory tests associated with Table II, identical amounts of Swine BlueLITE® and varying amounts of effervescent formula were combined to test the effect on the pH. Note that the pH increased less than 1 full point even when up to 30% of effervescent formula No. 1 (above) was added.

TABLE II

| | |
|---|---|
| pH of water without any SWINE | 7.33 |
| BLUELITE ® and EFFERVESCENT | |
| FORMULA | |
| pH of water with SWINE BLUELITE ® | 2.81 |
| and 1% EFFERVESCENT FORMULA | |
| pH of water with SWINE BLUELITE ® | 2.90 |
| and 3% EFFERVESCENT FORMULA | |
| pH of water with SWINE BLUELITE ® | 3.09 |
| and 5% EFFERVESCENT FORMULA | |
| pH of water with SWINE BLUELITE ® | 3.22 |
| and 10% EFFERVESCENT FORMULA | |
| pH of water with SWINE BLUELITE ® | 3.58 |
| and 20% EFFERVESCENT FORMULA | |
| pH of water with SWINE BLUELITE ® | 3.72 |
| and 30% EFFERVESCENT FORMULA | |

Similar results have been obtained utilizing bovine and avian acidified rehydration formulas. Table III shows a chart based on a present Bovine BlueLITE® formula. Whereas the pH with respect to the Bovine BlueLITE® formula with the effervescent composition appears to stabilize much sooner than the drinking water without the effervescent composition. Final differences are well within acceptable tolerances and within the margin of measurement error with respect to measuring the pH as explained below.

TABLE III

| | pH measurements | |
|---|---|---|
| | pH of water with | pH of water with no |
| | BOVINE | BOVINE BLUELITE ® or |
| | BLUELITE ® | EFFERVESCENT FORMULA |
| | 7.24 | 7.10 |
| | pH of water with | pH of water with |
| | BOVINE | BOVINE BLUELITE ® and |
| | BLUELITE ® | EFFERVESCENT FORMULA |
| pH after 10 seconds | 4.20 | 3.60 |
| with no stirring or | | |
| mixing action | | |
| pH after 20 seconds | 4.05 | 3.40 |
| with no stirring or | | |
| mixing action | | |
| pH after 30 seconds | 3.96 | 3.37 |
| with no stirring or | | |
| mixing action | | |
| pH after 40 seconds | 3.91 | 3.43 |
| with no stirring or | | |
| mixing action | | |
| pH after 50 seconds | 3.84 | 3.44 |
| with no stirring or | | |
| mixing action | | |
| pH after 1 minute | 3.81 | 3.47 |
| with no stirring or | | |
| mixing action | | |
| pH after 1½ | 3.79 | 3.46 |
| minutes with no | | |
| stirring or mixing | | |
| action | | |
| pH after 2 minutes | 3.81 | 3.50 |
| with no stirring or | | |
| mixing action | | |

It should be noted in the tests associated with the tables that the water solution of the formula was maintained in the identical dosage ratio as recommended for use to the producers of the animal species which, in the case of swine, was the addition of the ratio of 2 pounds of rehydrating powder to 128 gallons of drinking water.

In addition, when the pH of the water solutions was measured in accordance with the data reported in this application, the results were obtained with pH litmus paper or hand-held digital pH sensors. In this regard, the pH readings may vary from 0.10 to 1.00 points with respect to continuous subsequent readings measuring the same solutions. The variations in pH readings are attributed to variations in the sensitivity of pH litmus paper and tolerable differences in the recordings on the digital pH sensors. These variations are acceptable as normal variances by technicians in chemical labs, veterinary and human clinical laboratories. Thus, the differences in pH obtained with the acidified rehydration preparations alone or with the balanced effervescent compositions are well within the margin of error for the measurements.

This invention has been described herein in considerable detail in order to comply with the patent statutes and to provide those skilled in the art with the information needed to apply the novel principles and to construct and use embodiments of the example as required. However, it is to be understood that the invention can be carried out by specifically different devices and that various modifications can be accomplished without departing from the scope of the invention itself.

What is claimed is:

1. An effervescent acidified rehydration preparation for administration in the drinking water of mammals and poultry comprising:
(a) an amount of a dry generally water soluble acidified electrolyte composition for treatment of dehydration in animals including mammals and poultry by oral administration in the drinking water of animals of interest;
(b) an amount of a pH balanced effervescent composition comprising an amount of one or more compatible water soluble organic acid species and an amount of one or more water soluble effervescent agents; and
(c) wherein effervescence occurs immediately upon said rehydration preparation being added to water.

2. A effervescent acidified rehydration preparation as in claim 1 wherein said effervescent composition comprises from 1% to 30% (weight) of said rehydration preparation.

3. A effervescent acidified rehydration preparation as in claim 1 wherein said rehydration preparation includes an amount of citric acid.

4. A effervescent acidified rehydration preparation as in claim 1 wherein said pH balanced effervescent composition comprises organic acid species and water soluble effervescent agents in a ratio of approximately 1:2.

5. An effervescent acidified rehydration preparation as in claim 1 wherein said organic acid species of said effervescent composition is selected from the group consisting of citric acid, tartaric acid, malic acid, fumaric acid and combinations thereof.

6. An effervescent acidified rehydration preparation as in claim 1 wherein said water soluble effervescent agents are selected from sodium bisulfate, sodium bicarbonate, sodium carbonate, potassium carbonate, magnesium carbonate, calcium bicarbonate and combinations thereof.

7. An effervescent acidified rehydration preparation as in claim 6 wherein said water soluble effervescent agents are selected from sodium bisulfate, sodium bicarbonate, sodium carbonate, potassium carbonate, magnesium carbonate, calcium bicarbonate and combinations thereof.

8. An effervescent acidified rehydration preparation as in claim 7 wherein said effervescent composition includes an amount of sodium bicarbonate and an amount of citric acid.

9. An effervescent acidified rehydration preparation as in claim 4 wherein said effervescent composition includes an amount of sodium bicarbonate and an amount of citric acid.

10. A method of stabilizing a composition of water containing an acidified rehydration preparation for treatment of dehydration in animals including mammals and poultry designed for being orally administered in the drinking water of animals of interest comprising adding an effective amount of a pH balanced effervescent composition to said rehydration preparation to obtain thorough self-mixing of the preparation when dissolved in the drinking water of the animals of interest without adversely affecting the desired pH of the drinking water.
